(19) United States
(12) Patent Application Publication
(10) Pub. No.: US 2022/0105126 A1
(43) Pub. Date: Apr. 7, 2022

(54) COMPOSITION FOR PREVENTING AND TREATING MICROBIAL DISEASE

(71) Applicant: Craig Mercer Boulris, Danville, CA (US)

(72) Inventor: Craig Mercer Boulris, Danville, CA (US)

(21) Appl. No.: 17/492,451

(22) Filed: Oct. 1, 2021

Related U.S. Application Data
(60) Provisional application No. 63/086,988, filed on Oct. 2, 2020.

(51) Int. Cl. A61K 33/04; A61K 31/10; A61K 45/06; A61K 47/10

(52) U.S. Cl. CPC A61K 33/04; A61K 47/10; A61K 45/06; A61K 31/10

(57) ABSTRACT

A composition for preventing and treating microbial diseases may include dimethyl sulfide (DMS); dimethyl sulfoxide (DMSO); and elemental sulfur. The composition may also include methylsulfonylmethane, water, surfactants, terpenes, terpenoids, thioterpenols, bronchodilators, and yield enhancers. A method for preventing and treating microbial disease, viruses, fungi, bacteria, and parasites may include administering a prophylactically or therapeutically effective dosage of the composition to a patient in need.

### RELATED APPLICATION

This application claims priority to provisional patent application U.S. Ser. No. 63/086,988 filed on Oct. 2, 2020, the entire contents of which is herein incorporated by reference.

### BACKGROUND

The embodiments described herein relate generally to medical treatments and, more particularly, to a composition for preventing and treating microbial diseases.

Covid-19 (SARS-CoV-2) is, first and foremost, a respiratory disease. Covid-19 is a virus whose lesions typically infect the outer lung fields. The peripheral location makes Covid-19 more difficult to treat, due to the inaccessibility of small airways and alveoli, which can become thickened, inflamed, and congested. Covid-19 also effects small vascular structures and multiple other organ systems. Current therapeutics are inadequate at permeating these tissues and deactivating virions. Additionally, Covid-19 treatment can be complicated by insidious and difficult-to-treat pulmonary fungal infections adding to overall morbidity.

Current therapeutics are unable to effectively manage symptoms in patients who experience Covid-19 vaccine breakthrough or in the many unvaccinated individuals who acquire SARS-CoV-2. An unacceptable number of people are still hospitalized, remain incapacitated for months, are left with long-Covid symptoms, or expire. Societies still reel under fears of recurrent outbreaks. Tensions and economic damages related to mandates and distancing policies persist.

At some unpredictable point in the future, there is high probability of SARS-CoV-2 variant vaccine escape. Without an effective therapeutic, the world will revert back to square-one and need to reinitiate a new round of social distancing restrictions/quarantines, vaccine development, trials and distribution that might last 8-12 months, assuming new effective vaccines can even be developed. This cycle could repeat a number of times over the next decade, significantly impacting quality-of-life and economic conditions worldwide.

Therefore, what is needed is a composition that is both a prophylactic and a therapeutic and is a universal anti-microbial nano-drug combination that is effective in both pulmonary tissues and systematically against a broad spectrum of viruses (including Covid-19), fungi, bacteria, and parasites.

### SUMMARY

Some embodiments of the present disclosure include a composition for preventing and treating microbial diseases. The composition may include dimethyl sulfide (DMS); dimethyl sulfoxide (DMSO); and elemental sulfur. The composition may also include methylsulfonylmethane (MSM), water, surfactants, terpenes, terpenoids, thioterpenols, bronchodilators, and yield enhancers. A method for preventing and treating microbial disease, viruses, fungi, bacteria, and parasites may include administering a prophylactically or therapeutically effective dosage of the composition to a patient in need.

### DETAILED DESCRIPTION

In the following detailed description of the invention, numerous details, examples, and embodiments of the invention are described. However, it will be clear and apparent to one skilled in the art that the invention is not limited to the embodiments set forth and that the invention can be adapted for any of several applications.

The composition of the present disclosure may be used to prevent and treat microbial disease and may comprise the following elements. This list of possible constituent elements is intended to be exemplary only, and it is not intended that this list be used to limit the composition of the present application to just these elements. Persons having ordinary skill in the art relevant to the present disclosure may understand there to be equivalent elements that may be substituted within the present disclosure without changing the essential function or operation of the composition.

The various elements of the present disclosure may be related in the following exemplary fashion. It is not intended to limit the scope or nature of the relationships between the various elements and the following examples are presented as illustrative examples only.

By way of example, embodiments of the present disclosure include a composition and method for preventing and treating microbial diseases, the composition comprising dimethyl sulfide (DMS), dimethyl sulfoxide (DMSO), and elemental sulfur, such as micronized elemental sulfur. In some embodiments, the composition may also include methylsulfonylmethane (MSM), water, surfactants, terpenes/terpenoids/thioterpenols, bronchodilators, and yield enhancers. MSM may play a role as a yield enhancer or carrier. Bronchodilators may aid in pulmonary delivery. The composition may further comprise permeation enhancers, emollients, emulsifiers, and wetting agents. An exemplary wetting agent that may be included is propylene glycol or any other inert wetting agent. In embodiments, the composition may include an electrolyte solution or other diluent for DMS, which is flammable and, as such, administration in pure form may be problematic for storage, safety, delivery, and tissue irritation. Thus, the composition may comprise a combination of small-sized, dimethyl compounds with a central sulfur atom in a solvent.

Creating the composition of the present disclosure may include putting the components in a solution, gel, cream, or other suspension in the desired doses appropriate for mode of delivery and treatment purposes and then placing the combination in the desired delivery device, such as nasal or oral spray, nebulizer, metered dose inhaler, vape device, intravenous piggyback, dermal patch, or the like. In a vape device, the active ingredient, DMS may be heated to above its boiling point of 37° C./99° F. More specifically, making the composition may comprise providing a 2% DMS solution; dissolving micronized elemental sulfur 5 mg/mL in a solvent such as distilled water or an electrolyte solution; optionally adding a wetting agent, such as propylene glycol and, more specifically 10% propylene glycol, to the dissolved micronized elemental sulfur; mixing the DMS solution and the dissolved micronized elemental sulfur; adding DMSO in the desired concentration, such as 70% or less; optionally adding terpenes/terpenoids/thioterpenols to the mixture in an amount of, for example, up to about 1 to 2 metric drops per 100 mL for odor control.

A method for treating or preventing microbial diseases may include administering a prophylactic or therapeutic effective dosage of the composition to a patient in need. In embodiments, an effective dosage may comprise up to about 70% by volume (vol. %) DMSO, up to about 5 vol. % DMS, and up to about 40 g of elemental sulfur, based on a total volume of about 1000 mL. Concentrations and weight/weight of the active agents in the composition may need to be at the lower end of the range for administration through more sensitive tissues, such as the mucous membranes of the nasal passages and lungs. Less sensitive tissue, such as the dermis, may tolerate concentrations and weight/weight of active agents in the composition at higher range levels. Administering the composition may be performed topically, intravenously, via aerosolized liquid inhalation, via vaporized gas inhalation, by subcutaneous/intramuscular/other injection, orally, nasally, intraocularly, sublingually, rectally, via the bladder, intra-peritoneal or other catheter, by implantable device, by ophthalmologic/optic drops, by other known modes, or by a combination of modes. Heat/infrared iontophoresis, ultrasound/sonophoresis, laser, or other applications of heat/energy may also help facilitate delivery. Each mode of delivery may have its own pros and cons, meaning that the choice of delivery may be determined based on the disease being treated, the disease location and the individual patient. DMS has a boiling point of 37° C./99° F. It may be administered as a liquid at room temperature. DMS gradually becomes gaseous at human body temperature, wherein DMS may dissolve uniformly as a gas in blood, body fluids, and tissues. When administration of the composition is performed through inhalation, the DMS may be heated to its boiling point and inhaled in the form of a fully vaporized gas capable of penetrating deeper into air spaces than when used as an aerosolized liquid inhalant. The composition may also be delivered by IV, transdermal, or other routes to reach the circulation, resulting in an attack of peripheral lung lesions from the opposite side of the alveoli, via the pulmonary capillaries. Blood-borne active agents in the composition may also target microbes in non-pulmonary systems, tissues, and body fluids.

DMS has a molecular mass of 62 g and may be considered a nano-drug. DMS may be capable of diffusing across every fluid space and penetrating through every cell before ultimately crossing lung alveolar membranes and epithelial layers and being exhaled in breath. DMS may deactivate viruses everywhere along its course. DMS may circulate as a miscible liquid, and elemental sulfur a dissolved solid, prior to urinary excretion.

DMS undergoes no bioaccumulation, metabolism, degradation, or transformation along its entire course. As such, DMS may act as a safe, potent, whole body fumigant, performing its antiviral therapeutic function, then quickly leaving the body unchanged.

DMS may exert its antiviral therapeutic effects via a number of routes. First, DMS may act as a direct toxin, directly damaging and denaturing viral structural components, such as spike, membrane, and nucleo-capsid proteins or the viral envelope. DMS may inhibit or damage viral genes, including genes coding for spike proteins, wall components, or other structure proteins necessary for virion assembly. DMS may also inhibit viral RNA or DNA transcription or replication. Secondly, DMS may irreversibly bind to spike protein receptor-binding domains (RBDs) in a capping fashion, enveloping and sequestering virions, thereby blocking and disrupting their ability to attach to and infect host cells. Third, DMS, a known potent reducing agent, may function as a disulfide bond breaker. Disulfide bonding between cysteine residues in viral spike proteins and cysteine residues in host cellular membranes proteins facilitates viral attachment, fusion, and cell entry. DMS may effectively disrupt these cysteine-cysteine bonds, cleaving and detaching viruses attempting to bind and fuse to host cell membranes, and preventing infection.

DMS may act through a number of secondary pathways. DMS cleavage of cysteine-cysteine bonds in dermal and respirator epithelial keratin may mobilize free cysteine in blood. Through a number of cysteine catabolic pathways, cysteine is converted ultimately to hydrogen sulfide (H₂S) and other reactive sulfur species lethal to viruses. Supplemental elemental sulfur may favor formation of these reactive sulfur species. DMS may also act as a free-radical scavenger and antioxidant, functioning to mitigate the effects of inflammatory molecules generated both by viral infection and the body's immune response to infection, thereby protecting cells from further damage.

Lastly, DMS may be an immunologic gaseotransmitter, similar to hydrogen sulfide, acting to up-regulate a number of beneficial components or steps in immune system function or down-regulate components, like cytokines, or detrimental steps.

In vivo, dimethyl sulfoxide (DMSO) (CH₃)—SO—(CH₃) may undergo metabolism and disproportionation to dimethyl sulfide (DMS) (CH₃)—S—(CH₃) and methylsulfonylmethane (MSM) (CH₃)—SO₂—(CH₃). Therefore, DMSO may be used as a prodrug for DMS. Specifically, in the composition, DMSO plays a previously un-utilized role as an intentional, therapeutic delivery agent for its metabolite DMS. Historically, the odorous DMS was viewed as an unwanted contaminant and undesirable residue of both commercial DMSO synthesis and animal DMSO metabolism. For all prior medical DMSO usages, DMS presence was to be avoided if possible with additional distillation steps added to rid DMSO of DMS. In this composition and medical DMSO usage herein, however, DMSO may be included to purposely achieve the end goal of generating its metabolite, DMS. Generation of maximal free DMS is the objective, which may be facilitated by the addition of yield enhancers, such as elemental sulfur. DMSO, with a half-life measured in days, may be added to the composition as a long-acting DMS source, and accompany the composition's short-acting agent DMS, having a half-life measured in hours. In this composition, a secondary purpose for DMSO inclusion may be as a solvent/permeation enhancer/diluent. Moreover, DMSO may have some independent anti-microbial action of its own.

In the composition, the elemental sulfur may function as a yield and potency enhancer. Metals, such as copper, in blood proteins may bind sulfur components. Such binding is undesirable in the case of DMS/DMSO and will remove DMS from active circulation, preventing it from crossing into the lungs. Copper and other sulfur-binding metals, however, may have a greater affinity for elemental sulfur than for DMS. Thus, simultaneous elemental sulfur administration with DMS/DMSO may result in elemental sulfur blocking the sulfur-binding metals, preventing them from binding to DMS/DMSO. As such, more DMS will be available to exert its effect and, therefore, less DMS/DMSO may be used in the original medication to achieve the same result. The elemental sulfur may also function independently as an anti-fungal itself.

Some embodiments of the composition may be used prophylactically, wherein the composition may be provided over a prolonged period of time. Blood levels for prophylaxis may not need to be as high as those for treatment for active disease. In some embodiments, the prophylactic use may achieve measurable blood level of the composition for up to 24 hours and, thus, the prophylactic composition may be administered less frequently, for example, once per day.

Some embodiments of the composition may be used therapeutically for treatment of confirmed disease, wherein the composition may be used on an inpatient or outpatient basis and may be under the direct supervision of medical professionals. Blood levels for optimal treatment of known diseases may need to be higher than those for prophylaxis. Thus, the therapeutic composition may be administered more frequently, for example, more than once per day. Combined routes of administration may be necessary or beneficial.

The composition may demonstrate broad spectrum, anti-microbial action, wherein target microbes may include viruses, such as SARS, MERS and other Coronaviruses, Influenza A & B viruses, Parainfluenza virus, Respiratory Syncytial Virus and other Paramyxoviruses, Rhinovirus, Adenovirus, Ebola virus, Variola virus/Smallpox, Zika, Dengue/Yellow fever viruses, Rotavirus, Marburg virus, Rabies, Hantavirus, Lassa fever virus, Japanese encephalitis virus, Reovirus, Creutzfeldt-Jacob disease, HIV, Herpes Simplex Virus, Human Papilloma virus, Viral Meningitis, Tobacco Mosaic virus, Tomato Spotted Wilt virus, Tomato Yellow Leaf Curl Virus, Cucumber Mosaic virus, Potato Virus, Cauliflower Mosaic virus, African Cassava Mosaic virus, and other human, animal and plant viruses; fungi, such as Candida, Trichophyton, onychomycosis, Fusarium, Cryptococcus, Ajellomyces/Histoplasma Trichosporon Arthroderma, Microsporum, Epidermophyton, Blastomyces, Histoplasma, Paracoccidiod, Erysiphales, Eutypa, Anthracnose, Botrytis rots, Downy mildews, Powdery mildews, Rusts, Rhizoctonia rots, Sclerotinia rots, Sclerotium rots, Aspergillosis, Coccidioidomycoses, Peach Leaf Curl, Apple Scab and other human, animal and plant fungal infections; and bacteria and parasites, such as Tuberculosis, Typhus, Plague, Diphtheria, Typhoid, Vibrio cholerae/Cholera, Dysentery: Shigella, Campylobacter, Salmonella, or enterohemorrhagic E. coli, Pneumonia, Anthrax, Staph, Strep, Methicillin-Resistant Staph aureus (MRSA), P acnes, Bordetella pertussis, Clostridium tetani/Tetanus, Bacterial meningitis, Syphilis/Treponema pallidum, Neisseria gonorrhoeae, Measles/Rubeola, Leprosy/Mycobacterium leprae, and other human, animal and plant bacterial diseases, as well as Malaria/Plasmodium and other human, animal, and plant and parasitic diseases.

The composition may be capable of penetrating deep into the peripheral lung fields and systemically. The composition may effectively deactivate pulmonary pathogens by targeting Covid-19 at a number of different levels with positive cooperativity. Mulitfactorial mechanisms-of-action may increase DMS lethality to viruses, making it difficult for viral variants to mutate around the composition to achieve therapeutic escape. Use of the composition may effectively treat symptoms in current cases of vaccine breakthrough and in the unvaccinated who acquire the virus. In the event of vaccine escape, the composition may stand in the gap, until new vaccines can be developed and distributed. The composition may represent the offensive weapon that will reduce transmissibility, diminish the severity of viral infection, reduce numbers of hospitalization/intensive care unit stays and their durations, decrease co-morbid conditions, and limit mortality. In fact, the composition could render deadly microbial diseases, such as Covid-19, quickly and effectively treatable and no more dangerous than the common cold. Thus, the composition may ultimately limit the need of social distancing, mask and vaccine mandates, and permit resumption of normal societal and economic activity.

The main active ingredients are plentiful, inexpensive, high-production volume organosulfur compounds, wherein each has known toxicity, safety metabolism, tissue accumulation, and excretion profiles. Raw organosulfur compounds may readily be refined to medical grade for inclusion in the composition.

While the composition is described above as being effective for treating microbial disease in humans, it may also have veterinary antimicrobial applications and agricultural antimicrobial applications. It may also be an effective cleaner, disinfectant, fumigant, or decontaminant for hands, clothing, masks, filters, equipment, surfaces, physical spaced, structures, vehicles, municipal water supplies, food and food packaging, and the like.

The above-described embodiments of the invention are presented for purposes of illustration and not of limitation. While these embodiments of the invention have been described with reference to numerous specific details, one of ordinary skill in the art will recognize that the invention can be embodied in other specific forms without departing from the spirit of the invention. Thus, one of ordinary skill in the art would understand that the invention is not to be limited by the foregoing illustrative details, but rather is to be defined by the appended claims.

What is claimed is:

1. A composition for preventing and treating microbial diseases, the composition comprising:
dimethyl sulfide (DMS);
dimethyl sulfoxide (DMSO); and
elemental sulfur.

2. The composition of claim 1, further comprising at least one ingredient selected form the group consisting of methylsulfonylmethane, water, surfactants, terpenes, terpenoids, thioterpenols, bronchodilators, and yield enhancers.

3. The composition of claim 1, further comprising propylene glycol.

4. The composition of claim 1, wherein the elemental sulfur is micronized elemental sulfur.

5. The composition of claim 1, wherein the composition comprises, based on a total volume of about 1000 mL:
up to about 70 vol. % DMSO;
up to about 5 vol. % DMS; and
up to about 40 g of elemental sulfur.

6. The composition of claim 1, further comprising at least one member selected from the group consisting of a permeation enhancer, an emollients, an emulsifiers, and a wetting agent.

7. A method for treating or preventing microbial diseases, the method comprising:
administering a prophylactic or therapeutic effective dosage of a composition to a patient in need,
wherein the composition comprises:
dimethyl sulfide (DMS);
dimethyl sulfoxide (DMSO); and
elemental sulfur.

8. The method of claim 7, wherein administering the composition comprises a member selected from the group consisting of topical administration, intravenous administration, aerosolized liquid inhalation, vaporized gas inhalation, injection, oral administration, nasal administration, intraocular administration, sublingual administration, rectal administration, catheter administration, administration by implantable device, administration by ophthalmologic/optic drops, and a combination thereof

9. The method of claim 7, further comprising applying heat/energy during administering the dosage.

10. The method of claim 7, wherein:
administering the dosage comprises providing the composition through an inhaled method; and
the method further comprises heating the DMS to its boiling point prior to administration to provide a fully vaporized gas.
